(19) United States
(12) Patent Application Publication
(10) Pub. No.: US 2004/0242957 A1
(43) Pub. Date: Dec. 2, 2004

(54) DEVICE FOR ALLEVIATING ERECTILE DYSFUNCTION

(76) Inventor: Raymond A. Ward, Beasley, TX (US)

Correspondence Address: Richard C. Litman, LITMAN LAW OFFICES, LTD., P.O. Box 15035, Arlington, VA, 22215, US

(21) Appl. No.: 10/445,795

(22) Filed: May 28, 2003

(51) Int. Cl. A61F 5/00

(52) U.S. Cl. 600/39

(57) ABSTRACT

The device for alleviating erectile dysfunction is a malleable band with a single gap that is secured partially around the base of a human penis to restrict the flow of blood out of the penis. The band is easily shaped for a proper fit by applying finger pressure thereto, yet is sufficiently rigid to hold its shape under pressure from an expanding or erect penis. The gap measures approximately one-half inch, and is positioned on the inferior side of the penis such that the device does not constrict the urethra. The device is reusable and can be easily applied to or removed from an erect penis during intercourse without necessitating withdrawal of the penis.

### BACKGROUND OF THE INVENTION

1. Field of the Invention

The present invention relates to devices for assisting human males suffering from erectile dysfunction in maintaining a penile erection for sexual intercourse and, more particularly, to a device secured around the base of the penis to restrict the flow of blood out of the penis.

2. Description of the Related Art

According to the National Institute of Health, approximately 30 million men in America suffer from erectile dysfunction or impotence. Erectile dysfunction is a condition where a male is not capable of attaining or sustaining an erect penis that is sufficiently rigid for sexual intercourse. It is a problem especially common in older males—half of the 30 million men suffering from erectile dysfunction are thought to be over age 65, and five sixths over age 40. Furthermore, at age 65, erectile dysfunction is thought to occur in one of every four males.

Generally, an erection is caused by an imbalance of blood flow to and from the penis. Arterial blood flows freely into the penis while venous blood flowing out of the penis is restricted, which thereby causes the penis to become engorged. The restriction in venous blood flow is normally caused by sphincter muscles that respond to sexual stimuli.

It is believed that between 60 and 80% of men experiencing erectile dysfunction due so as a result of venous leakage. Venous leakage occurs when the flow of blood out of the penis is not sufficiently restricted during the erectile process, thereby resulting in excessive drainage of blood and subsequent detumescence or erectile failure.

A number of therapies for addressing erectile dysfunction are known. One therapy is vaso-surgery to deaden or sever penile veins. Another therapy is surgical implantation of either rubber rods or inflatable silicone vessels into the penis. However, it is believed that most men consider these surgical procedures to be highly invasive.

Another therapy is the use of Viagra® (a trademark of Pfizer) or other similar drugs that assist males in achieving and maintaining an erection. The use of Viagra, however, has two well-known drawbacks. First, men who are also taking medication for heart ailments cannot use the drug. This is a drawback because many of the men who suffer from erectile dysfunction are of an age associated with the occurrence of heart ailments and, hence, take such medication. Second, Viagra is effective approximately thirty minutes after consumption. Thus, due to its thirty minute lag time, Viagra may inhibit spontaneity or, if the female partner is unable or unwilling to wait thirty minutes, may not be a viable option.

Another therapy is the manual restriction of venous blood flow out of the penis. Because penile arteries are located deep within the penis, whereas penile veins are disposed near the surface of the penis, a number of devices are known which restrict blood flow out of the penis by applying pressure to the surface of the penis around its base. These devices can be categorized into one of two groups: one group consisting of ring or ring-like devices, and a second group consisting of closable devices that are secured around a penis via a clasp or other mechanism. However, as discussed below, each of these devices has at least one drawback.

Examples of ring or ring-like devices include the devices shown in U.S. Pat. No. 4,995,381 to Marmar et al.; U.S. Pat. No. 5,306,227 to Osbon et al.; U.S. Pat. No. 5,628,329 to Bennett et al.; U.S. Pat. No. 5,873,813 to Weiss; U.S. Pat. No. 6,306,080 to Mitchell et al.; and U.S. Pat. No. 6,319,194 to Wulf. Each of these devices incorporates a continuous ring of a fixed size that is applied by inserting the tip of the penis into the ring and then sliding the ring from the tip of the penis to the base of the penis. As a result, all of these devices share three common drawbacks. First, because the devices are applied by being slid down the length of the penis, applying the devices can be time consuming and difficult. This is primarily due to the fact that each device is sized to fit snugly around the base of the penis, yet is typically applied when the penis is erect or partially swollen. Second, because the devices must fit snugly around the penis, typically a user must be sized by a urologist or other health care professional, which can be costly and embarrassing. Third, because these devices are meant to fit snugly around the penis and are difficult to remove during intercourse, they typically impede the flow of ejaculatory fluids which travel through a vessel near the surface of the penis.

Examples of closable devices that are secured around a penis via a clasp or other mechanism include the devices shown in U.S. Pat. No. 4,203,432 to Koch; U.S. Pat. No. 4,834,115 to Stewart; U.S. Pat. No. 5,027,800 to Rowland; U.S. Pat. No. 5,439,007 to Fischer; and U.S. Pat. No. 5,855,548 to Place; and U.S. Pat. Publication Nos. 2002/0024536 and 2002/0137983. These devices consist of tubing or an open ring-like member that is secured around the base of the penis with some form of a clasping mechanism. Each of the devices in this group is also subject to three drawbacks. First, none of these devices are capable of being shaped to fit the individual user while not restricting flow of ejaculatory fluid through the urethra. Second, a user of any of these devices must manipulate the clasping mechanism, a task that is not easily accomplished if attempted during intercourse. Third, none of these devices assimilate the male anatomy by resting flush or almost flush with the surface of the penis. As a result, each of these devices can be visually distracting to the user and his partner, and also can bump against sensitive areas of the user's partner during intercourse.

In addition to the devices discussed above, U.S. Pat. No. 5,192,271 to Kalb discloses a device for administering a stimulant to a flaccid penis. Although the device bares a faint structural resemblance to the present invention, the Kalb device differs from the present invention in several significant respects. First, given that the Kalb patent claims a device that is “flexible so that the circumference of the ring can be manually increased by . . . expansion of the penis,” the device would not be well-suited for restricting venous blood flow out of the penis during intercourse. Second, because the device does not rest flush with the surface of the penis, it can bump the female partner during intercourse. Third, because the device includes either a tab or a needle guide protruding from its outer surface, the tab or needle guide is especially prone to bump the female partner during intercourse. Fourth, the edges of the device are squared, which makes contact with the device uncomfortable for a female partner. And fifth, the device is comprised of more than one element, which increases the complexity of its manufacture and use.

Consequently, none of the above inventions and patents, taken either singly or in combination, is seen to describe the instant invention as claimed and, therefore, a device for alleviating erectile dysfunction solving the aforementioned problems is desired.

### SUMMARY OF THE INVENTION

The device for alleviating erectile dysfunction is a malleable band having a single gap defined therein that is secured around the base of a human penis to restrict the flow of blood out of the penis. The band is easily shaped for a proper fit by applying finger pressure thereto, yet is sufficiently rigid to hold its shape under pressure from an expanding or erect penis. The gap, which may measure approximately one-half inch, is positioned on the underside of the penis such that the device does not constrict the urethra. The device is reusable and can be easily applied to or removed from an erect penis during intercourse.

Accordingly, it is a principal object of the invention to provide a device that alleviates erectile dysfunction by applying pressure to the subcutaneous veins of the penis and thereby reducing the flow of venous blood out of the penis.

It is another object of the invention to provide a device which alleviates erectile dysfunction while avoiding the application of excessive pressure to the urethra which might otherwise impede the flow of ejaculatory fluids.

It is a further object of the invention to provide a device for alleviating erectile dysfunction that can be easily shaped for a proper fit using finger pressure.

Still another object of the invention is to provide a device for alleviating erectile dysfunction that can easily be applied to or removed from a penis during intercourse, without necessitating withdrawal of the penis, by first bending the device open and then bending it closed.

It is an object of the invention to provide improved elements and arrangements thereof for the purposes described which is inexpensive, dependable and fully effective in accomplishing its intended purposes.

These and other objects of the present invention will become readily apparent upon further review of the following specification and drawings.

### BRIEF DESCRIPTION OF THE DRAWINGS

FIG. 1 is an environmental, perspective view of a device for alleviating erectile dysfunction according to the present invention.

FIG. 2 is an environmental, side elevation view of the device for alleviating erectile dysfunction according to the present invention.

FIG. 3 is a perspective view of the device for alleviating erectile dysfunction according to the present invention.

FIG. 4 is a front view of the device for alleviating erectile dysfunction according to the present invention.

FIG. 5 is a top view of the device for alleviating erectile dysfunction when pressed flat.

FIG. 6 is a top view of an alternative embodiment of the device for alleviating erectile dysfunction when pressed flat

Similar reference characters denote corresponding features consistently throughout the attached drawings.

### DETAILED DESCRIPTION OF THE PREFERRED EMBODIMENTS

The present invention is a device for alleviating erectile dysfunction that is secured around a portion of the base of a human penis to restrict the flow of blood out of the penis by the application of pressure to the subcutaneous penile veins.

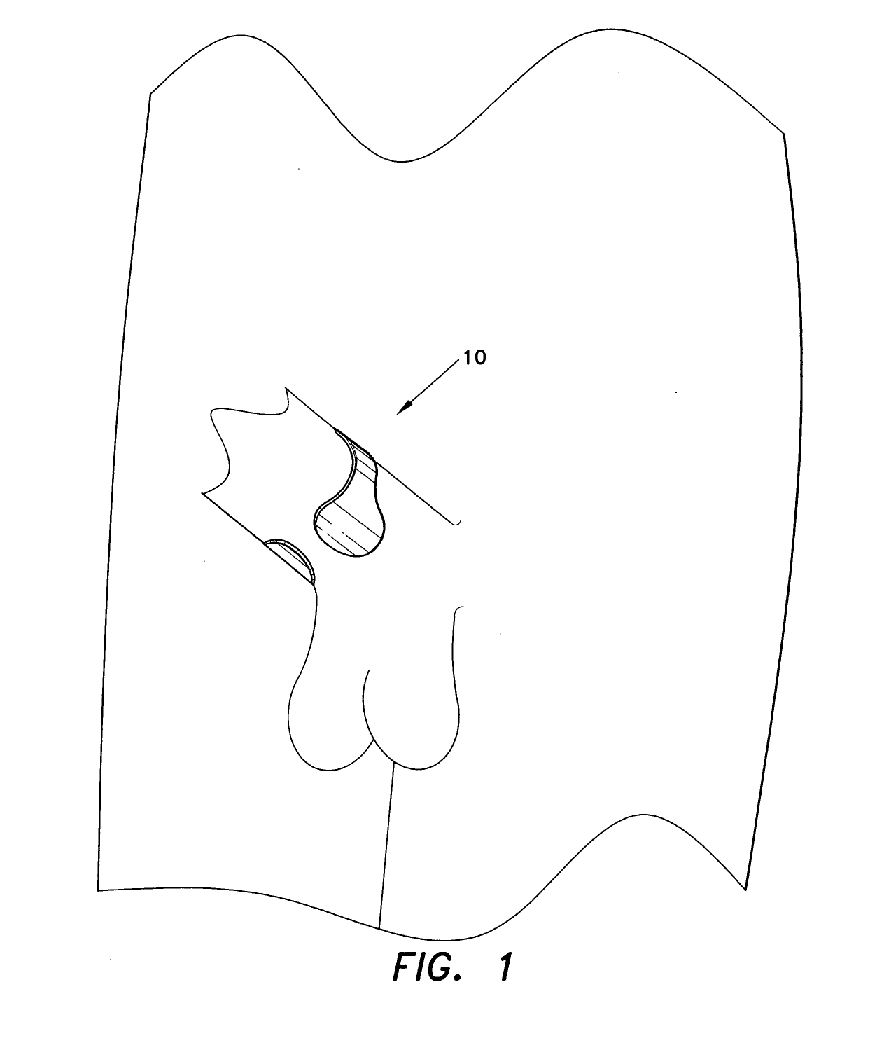

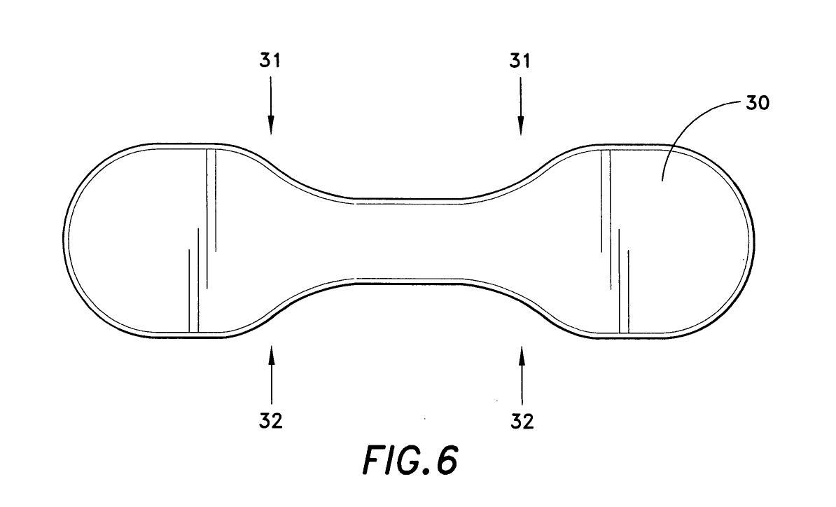

Referring to the drawings, FIGS. 1-5 illustrate a first embodiment of the device and FIG. 6 illustrates a slightly modified alternative embodiment of the device.

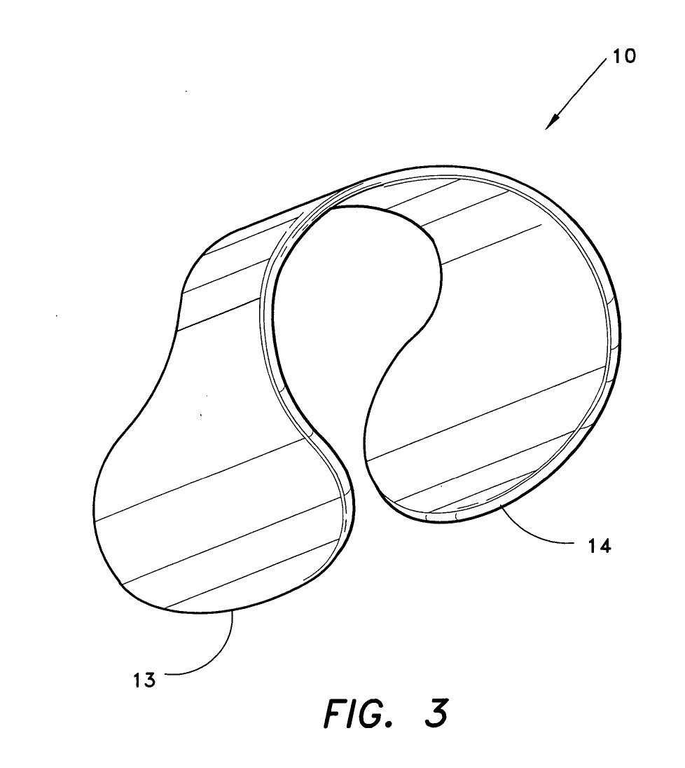

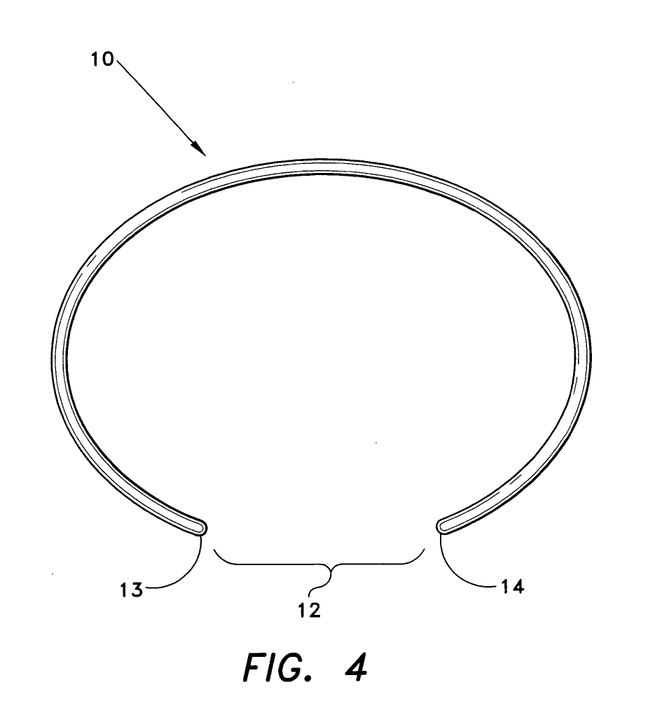

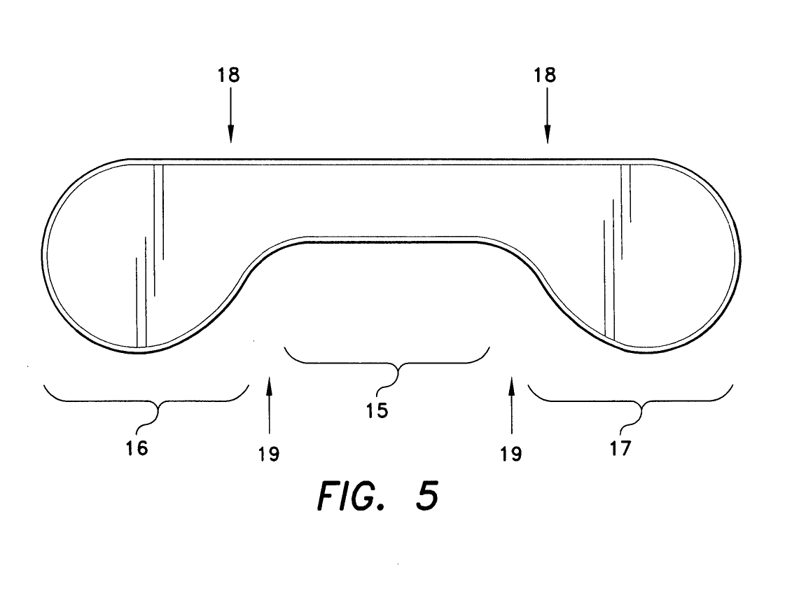

The device 10 consists of a band having a single gap 12 defined therein, as shown in FIGS. 3 and 4. The band forms an elliptical opening for receiving a penis. Each of the two ends 13 and 14 that define the gap 12 is formed by a rearward extending arcuate lobe having a curvature of about one-half inch in radius. The distance between the ends 13 and 14 measures about one-half inch but can be adjusted by finger pressure from about one-quarter of an inch to about three-quarters of an inch thereby allowing for an individualized fit based on the size of the penis. As shown in FIG. 5, which illustrates the device as pressed flat, the width of the band is not uniform. The middle segment of the band 15 is formed as a rectangular strip which is about ⅜ of an inch wide. The middle segment is narrower than the width of the lobes 16 and 17, which extend up to about one inch in width. From the perspective shown in FIG. 5, the front edge 18 of the device 10 is substantially straight, while the back edge 19 is curved to form lobes 16 and 17, i.e., the front edge of lobes 16 and 17 is substantially linear and continuous with the front edge of middle segment 15, while the rear edge of the lobes is arcuate.

The device 10 is constructed from a single piece of aluminum alloy that is easily shaped by finger pressure, but that holds its shape under pressure from an expanding or erect penis, such as an aluminum alloy known to those of ordinary skill in the art as 5052H-32 with a thickness of five-hundredths (0.05) of an inch. All of the device's edges are rounded. To inhibit oxidation of the device 10, the device 10 can be either anodized or coated with alodyne. The simple one-piece design serves to minimize manufacturing costs.

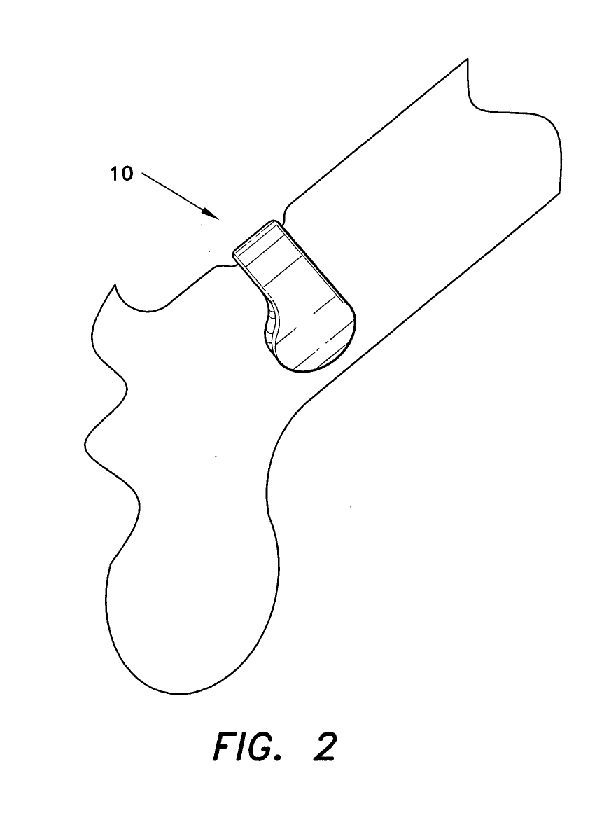

The device 10 is secured to the base of the penis as shown in FIGS. 1 and 2, either by spreading open its two ends 13 and 14 using finger pressure and then closing the device 10 around the penis, or by inserting the tip of the penis into the device 10 and then sliding the device 10 down the length of the penis. The device 10 is secured to the penis with the gap 12 positioned on the inferior side of the penis such that the device 10 does not contact or constrict the urethra and, thus, does not restrict the flow of ejaculatory fluids.

Because the device 10 has rounded edges and rests flush on the penis, as shown in FIGS. 1 and 2, it 10 can be used during sexual intercourse without discomfort to the partner. Hence, the user does not have to limit penetration during intercourse, as may be required by other devices which do not fit flush on the penis due to greater thickness or a more rigidly defined annular shape than the device 10 of the present invention.

Furthermore, since the device is only three-eighths of an inch in width with the lobes 16 and 17 extending rearward to the scrotum, the user can avoid contacting his partner with the device 10 altogether with a minimal reduction in the depth of penetration, if desired.

FIG. 6 depicts, as pressed flat, an alternative embodiment 30 of the present invention which differs from the preferred embodiment in only one respect. In this embodiment the lobes 16 and 17 are circular so that the front edge 31 curves forward at the lobes 16 and 17 so that the front edge 31 and the rear edge 32 are symmetrical. Thus, when pressed flat, the alternative embodiment 30 is symmetrical across both its horizontal and vertical axes.

It is to be understood that the present invention is not limited to the embodiments described above, but encompasses any and all embodiments within the scope of the following claims.

1. A device for alleviating erectile dysfunction, comprising:
a band having a opposing ends defining a gap between the opposing ends, the band being adapted for encircling a base of a penis with the gap straddling a urethra extending through the penis in order to constrict venous blood flow without restricting fluid flow through the urethra;
said band having a front edge and a rear edge, the opposing ends of said band being arcuate, rearward extending lobes defining a middle segment between the lobes;
said band being made from a thin strip of malleable material capable of being shaped by a person's fingers applying pressure thereto.

2. The device according to claim 1, wherein said band is constructed of a metal alloy.

3. The device according to claim 1, wherein said band is made from aluminum alloy 5052H-32.

4. The device according to claim 1, wherein said band is about 0.050 inches thick.

5. The device according to claim 1, wherein said band is made from aluminum and coated with alodyne.

6. The device according to claim 1, wherein said band is made from anodized aluminum.

7. The device according to claim 1, wherein said band is flesh colored.

8. The device according to claim 1, wherein the gap defined in said band is about one-half inch.

9. The device according to claim 1, wherein said rearward extending lobes do not extend past the front edge of said band.

10. The device according to claim 1, wherein said middle segment is an elongated rectangular strip about ⅜ of an inch in width.

11. (Cancelled)

12. A device for alleviating erectile dysfunction, comprising:
a band having a opposing ends defining a gap between the opposing ends, the band being adapted for encircling a base of a penis with the gap straddling a urethra extending through the penis in order to constrict venous blood flow without restricting fluid flow through the urethra;
said band has a front edge and a rear edge, the opposing ends of said band being arcuate, circular lobes and defining a middle segment between the lobes the front edge and the rear edge being symmetrical;
said band being made from a thin strip of malleable material capable of being shaped by a person's fingers applying pressure thereto.

13. The device according to claim 12,
wherein said band being constructed of a metal alloy; and
wherein said band being about 0.050 inches thick.

14. The device according to claim 12, wherein said band is made from aluminum alloy 5052H-32.

15. The device according to claim 12, wherein said band is made from aluminum and coated with alodyne or from anodized aluminum.

16. The device according to claim 12, wherein said band is flesh colored.

17. The device according to claim 12, wherein said middle segment is an elongated rectangular strip about ⅜ of an inch in width.

18. A device for alleviating erectile dysfunction, consisting of:
a band having a opposing ends defining a gap between the opposing ends, the band being adapted for encircling a base of a penis with the gap straddling a urethra extending through the penis in order to constrict venous blood flow without restricting fluid flow through the urethra;
said band has a front edge and a rear edge, the opposing ends of said band being arcuate, rearward extending lobes defining a middle segment between the lobes, said rearward extending lobes not extending past sail front edge.

19. The device according to claim 18,
wherein said band being constructed of a metal alloy; and
wherein said band being about 0.050 inches thick.

20. The device according to claim 12, wherein said rearward extending lobes do not extend past the front edge of said band.

21. The device according to claim 12, wherein said band is flesh colored.
